(19) United States
(12) Patent Application Publication
(10) Pub. No.: US 2010/0132062 A1
(43) Pub. Date: May 27, 2010

(54) Rice Mutant Allele

(76) Inventors: Christopher W. Deren, Almyra, AR (US); Karen A. K. Moldenhauer, Stuttgart, AR (US); Steven Brooks, Stuttgart, AR (US); Wengui Yan, Stuttgart, AR (US)

Correspondence Address: JONDLE & ASSOCIATES P.C., 858 HAPPY CANYON ROAD SUITE 230, CASTLE ROCK, CO, 80108, US

(21) Appl. No.: 12/625,329

(22) Filed: Nov. 24, 2009

Related U.S. Application Data
(60) Provisional application No. 61/200,271, filed on Nov. 26, 2008.

(51) Int. Cl. A01H 5/00; A01H 1/02; C07H 21/04; C07K 14/415; C12N 5/10; C12Q 1/68

(52) U.S. Cl. 800/260; 800/320.2; 536/23.6; 530/370; 435/419; 435/6

(57) ABSTRACT

A rice mutant allele designated Rc-g is disclosed. The invention relates to the Rc-g nucleotide sequence, the amino acid sequence, rice seeds containing the mutant allele Rc-g, to rice plants containing the mutant allele Rc-g and to methods for producing a rice plant containing the mutant allele Rc-g produced by crossing a rice plant containing allele Rc-g with itself or another rice variety. The invention further relates to hybrid rice seeds and hybrid rice plants containing mutant allele Rc-g.

This application claims the benefit of U.S. Provisional Application No. 61/200,271 filed Nov. 26, 2008 hereby incorporated by reference in its entirety.

### BACKGROUND OF THE INVENTION

This invention relates to a rice plant, seed, variety and hybrid. More specifically, the present invention relates to a rice plant mutant allele designated “Rc-g”. All publications cited in this application are herein incorporated by reference.

Rice is an ancient agricultural crop and is today one of the principal food crops of the world. There are two cultivated species of rice: Oryza sativa L., the Asian rice, and O. glaberrima Steud., the African rice. O. sativa L. constitutes virtually all of the world's cultivated rice and is the species grown in the United States. Three major rice producing regions exist in the United States: the Mississippi Delta (Arkansas, Mississippi, northeast Louisiana, southeast Missouri), the Gulf Coast (southwest Louisiana, southeast Texas), and the Central Valleys of California.

Rice in the United States is classified into three primary market types by grain size, shape, and chemical composition of the endosperm: long-grain, medium grain and short-grain. Typical U. S. long-grain cultivars cook dry and fluffy when steamed or boiled, whereas medium-and short-grain cultivars cook moist and sticky. Long-grain cultivars have been traditionally grown in the southern states and generally receive higher market prices.

There are numerous steps in the development of any novel, desirable plant germplasm. Plant breeding begins with the analysis and definition of problems and weaknesses of the current germplasm, the establishment of program goals, and the definition of specific breeding objectives. The next step is selection of germplasm that possess the traits to meet the program goals. The goal is to combine in a single variety an improved combination of desirable traits from the parental germplasm. These important traits may include pigmentation of the pericarp, higher seed yield, resistance to diseases and insects, better stems and roots, tolerance to low temperatures, and better agronomic characteristics on grain quality.

Choice of breeding or selection methods depends on the mode of plant reproduction, the heritability of the trait(s) being improved, and the type of cultivar used commercially (e.g., F₁ hybrid cultivar, pureline cultivar, etc.). For highly heritable traits, a choice of superior individual plants evaluated at a single location will be effective, whereas for traits with low heritability, selection should be based on mean values obtained from replicated evaluations of families of related plants. Popular selection methods commonly include pedigree selection, modified pedigree selection, mass selection, and recurrent selection, or a combination of these methods.

The complexity of inheritance influences choice of the breeding method. Backcross breeding is used to transfer one or a few favorable genes for a highly heritable trait into a desirable cultivar. This approach has been used extensively for breeding disease-resistant cultivars. Various recurrent selection techniques are used to improve quantitatively inherited traits controlled by numerous genes. The use of recurrent selection in self-pollinating crops depends on the ease of pollination, the frequency of successful hybrids from each pollination, and the number of hybrid offspring from each successful cross.

Each breeding program should include a periodic, objective evaluation of the efficiency of the breeding procedure. Evaluation criteria vary depending on the goal and objectives, but should include gain from selection per year based on comparisons to an appropriate standard, overall value of the advanced breeding lines, and number of successful cultivars produced per unit of input (e.g., per year, per dollar expended, etc.).

Promising advanced breeding lines are thoroughly tested and compared to appropriate standards in environments representative of the commercial target area(s) for three or more years. The best lines are candidates for new commercial cultivars; those still deficient in a few traits may be used as parents to produce new populations for further selection.

The goal of rice plant breeding is to develop new, unique and superior rice cultivars and hybrids. The breeder initially selects and crosses two or more parental lines, followed by self-pollination and selection, producing many new genetic combinations. The breeder can theoretically generate billions of different genetic combinations via crossing, selfing and mutations. The breeder has no direct control at the cellular level. Therefore, two breeders will never develop the same line, or even very similar lines, having the same rice traits.

Each year, the plant breeder selects the germplasm to advance to the next generation. This germplasm is grown under unique and different geographical, climatic and soil conditions and further selections are then made, during and at the end of the growing season. The cultivars which are developed are unpredictable. This unpredictability is because the breeder's selection occurs in unique environments, with no control at the DNA level (using conventional breeding procedures), and with millions of different possible genetic combinations being generated. A breeder of ordinary skill in the art cannot predict the final resulting lines he develops, except possibly in a very gross and general fashion. The same breeder cannot produce the same cultivar twice by using the exact same original parents and the same selection techniques. This unpredictability results in the expenditure of large amounts of research monies to develop superior new rice cultivars.

The development of new rice cultivars requires the development and selection of rice varieties, the crossing of these varieties and selection of superior hybrid crosses. The hybrid seed is produced by manual crosses between selected male-fertile parents or by using male sterility systems. These hybrids are selected for certain single gene traits such as semi-dwarf plant type, pubescence, awns, and apiculus color which indicate that the seed is truly a hybrid. Additional data on parental lines, as well as the phenotype of the hybrid, influence the breeder's decision whether to continue with the specific hybrid cross.

Pedigree breeding and recurrent selection breeding methods are used to develop cultivars from breeding populations. Breeding programs combine desirable traits from two or more cultivars or various broad-based sources into breeding pools from which cultivars are developed by selfing and selection of desired phenotypes. The new cultivars are evaluated to determine which have commercial potential.

Pedigree breeding is used commonly for the improvement of self-pollinating crops. Two parents which possess favorable, complementary traits are crossed to produce an F₁. An F₂ population is produced by selfing one or several F₁'s. Selection of the best individuals may begin in the F₂ population; then, beginning in the F₃, the best individuals in the best families are selected. Replicated testing of families can begin in the F₄ generation to improve the effectiveness of selection for traits with low heritability. At an advanced stage of inbreeding (i.e., F₆ and F₇), the best lines or mixtures of phenotypically similar lines are tested for potential release as new cultivars.

Mass and recurrent selections can be used to improve populations of either self- or cross-pollinating crops. A genetically variable population of heterozygous individuals is either identified or created by intercrossing several different parents. The best plants are selected based on individual superiority, outstanding progeny, or excellent combining ability. The selected plants are intercrossed to produce a new population in which further cycles of selection are continued.

Backcross breeding has been used to transfer genes for a simply inherited, highly heritable trait into a desirable homozygous cultivar or inbred line which is the recurrent parent. The source of the trait to be transferred is called the donor parent. The resulting plant is expected to have the attributes of the recurrent parent (e.g., cultivar) and the desirable trait transferred from the donor parent. After the initial cross, individuals possessing the phenotype of the donor parent are selected and repeatedly crossed (backcrossed) to the recurrent parent. The resulting plant is expected to have the attributes of the recurrent parent (e.g., cultivar) and the desirable trait transferred from the donor parent.

The single-seed descent procedure in the strict sense refers to planting a segregating population, harvesting a sample of one seed per plant, and using the one-seed sample to plant the next generation. When the population has been advanced from the F₂ to the desired level of inbreeding, the plants from which lines are derived will each trace to different F₂ individuals. The number of plants in a population declines each generation due to failure of some seeds to germinate or some plants to produce at least one seed. As a result, not all of the F₂ plants originally sampled in the population will be represented by a progeny when generation advance is completed.

In a multiple-seed procedure, rice breeders commonly harvest one or more seeds from each plant in a population and thresh them together to form a bulk. Part of the bulk is used to plant the next generation and part is put in reserve. The procedure has been referred to as modified single-seed descent or the pod-bulk technique.

The multiple-seed procedure has been used to save labor at harvest. It is considerably faster to thresh panicles with a machine than to remove one seed from each by hand for the single-seed procedure. The multiple-seed procedure also makes it possible to plant the same number of seeds of a population each generation of inbreeding. Enough seeds are harvested to make up for those plants that did not germinate or produce seed.

Descriptions of other breeding methods that are commonly used for different traits and crops can be found in one of several reference books (e.g., Allard, 1960; Simmonds, 1979; Sneep et al., 1979; Fehr, 1987).

Proper testing should detect any major faults and establish the level of superiority or improvement over current cultivars. In addition to showing superior performance, there must be a demand for a new cultivar that is compatible with industry standards or which creates a new market. The introduction of a new cultivar will incur additional costs to the seed producer, the grower, processor and consumer; for special advertising and marketing, altered seed and commercial production practices, and new product utilization. The testing preceding release of a new cultivar should take into consideration research and development costs as well as technical superiority of the final cultivar. For seed-propagated cultivars, it must be feasible to produce seed easily and economically.

Rice, Oryza sativa L., is an important and valuable field crop. Thus, a continuing goal of rice plant breeders is to develop stable, high yielding rice cultivars that are agronomically sound. The reasons for this goal are obviously to maximize the amount of grain produced on the land used and to supply food for both animals and humans. To accomplish this goal, the rice breeder must select and develop rice plants that have the traits that result in superior cultivars.

The foregoing examples of the related art and limitations related therewith are intended to be illustrative and not exclusive. Other limitations of the related are will become apparent to those of skill in the art upon a reading of the specification.

### BRIEF DESCRIPTION OF THE FIGS.

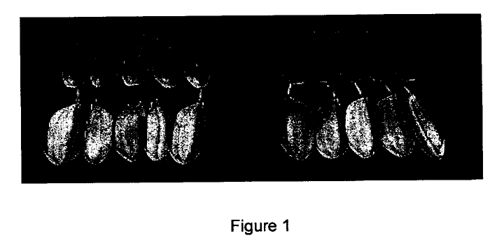

FIG. 1. Close-up images of Wells (left) and Red Wells (right); de-hulled seed (top) and ‘paddy rice’ bottom.

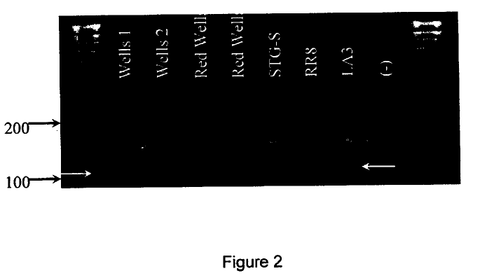

FIG. 2. PCR amplification with the RID12 (Sweeney, et al., 2006) marker. Rice cultivar Wells (rc; lanes 2 and 3), Red Wells mutant (Rc-g; lanes 4 and 5), red rice accession STG-S (Rc; lane 6), red rice accession RR8 (Rc; lane 7), and red rice accession LA3 (Rc; lane 8). Negative (no template DNA) control in lane 9. Size standard HyperLadder IV (Bioline, Randolph, Mass.), with 100 and 200 by markers indicated at left (lanes 1 and 10). RID12 target fragment indicated by white arrows. Fragments resolved on 2.3% METAPHORE agarose (Cambrex, Rockland, Me.).

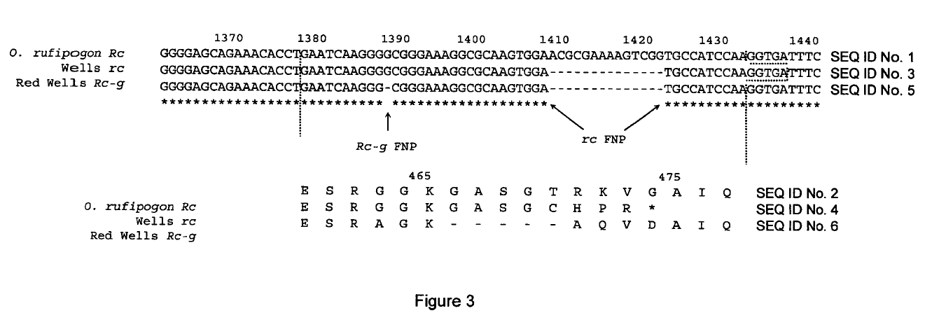

FIG. 3. DNA and putative protein sequence alignments of Rc, rc, and Rc-g alleles. Rc, SEQ ID NO: 1 (DNA) and SEQ ID NO: 2 (Protein) (O. rufipogon cv. IRGC105491; DQ204737; Sweeney, et al., 2006), rc, SEQ ID NO: 3 (DNA) and SEQ ID NO: 4 (Protein) (O. sativa cv. Wells), and Rc-g, SEQ ID NO: 5 (DNA) and SEQ ID NO: 6 (Protein) (O. sativa mutant Red Wells). The Rc-g 1 bp-FNP at position 1388 and the rc 14bp-FNP (positions 1408-1421) are indicated by arrows. The region of DNA sequence translation is delineated by dashed lines. * indicates a premature stop codon in rc. Deleted nucleotide and amino acid positions are filled with dashes (-) to justify sequence alignments. Nucleotide and amino acid positions (O. rufipogon; Rc) are indicated above the alignments.

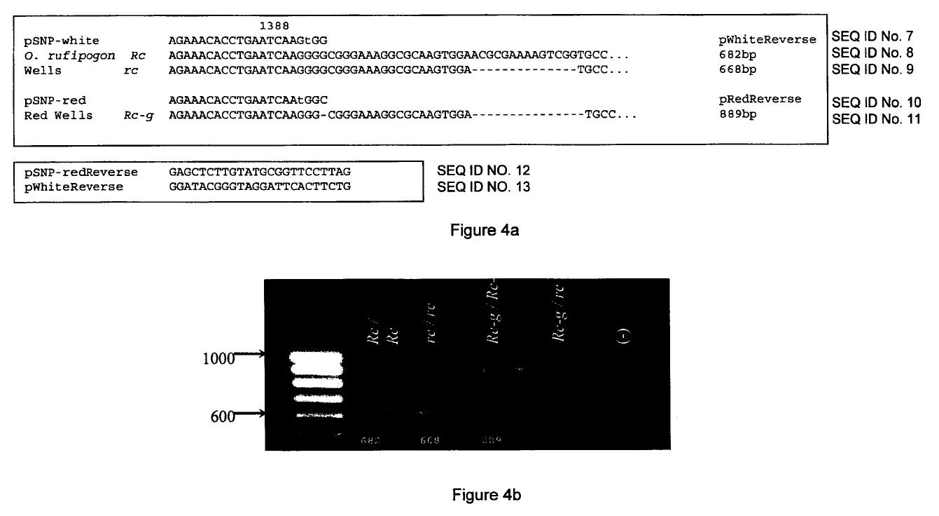

FIG. 4a. SNP primer alignments to corresponding DNA target sequences of Rc, rc, and Rc-g alleles. Rc, SEQ ID NO: 8 (O. rufipogon cv. IRGC105491; DQ204737; Sweeney, et al., 2006), rc, SEQ ID NO: 9 (O. sativa cv. Wells), and Rc-g, SEQ ID NO: 11 (O. sativa mutant Red Wells). The Rc-g 1 bp-FNP at position 1388 and the rc 14bp-FNP (positions 1408-1421) are indicated by dashes. Amplicon sizes for primer pairs are indicated at right of the sequences.

FIG. 4b. PCR amplification with the RCG-FNP marker. Size standard HyperLadder IV (Bioline, Randolph, Mass.), with 600 and 1000 by markers indicated at left (lane 1), and band sizes in by indicated below lanes 2-4. Red rice accession STG-S (lane 2), rice cultivar Wells (lane 3), Red Wells mutant (lane 4), and a F₁ from the Red Wells/Wells cross (lane 5). Negative (no template DNA) control in lane 6. Fragments resolved on 1.5% agarose (Bio-Rad, Hercules, Calif.).

### SUMMARY OF THE INVENTION

The following embodiments and aspects thereof are described in conjunction with systems, tools and methods which are meant to be exemplary and illustrative, not limiting in scope. In various embodiments, one or more of the above described problems have been reduced or eliminated, while other embodiments are directed to other improvements.

A rice mutant allele, designated “Rc-g”, is described for production of red pericarp and red seed coat in rice. A rice seed, a rice plant, a rice cultivar, and a rice hybrid are also described, all containing the Rc-g allele. The mutant allele is also described in plants, seeds, and other plant parts such as pollen and ovules. In addition, the Rc-g allele may be transfered to other rice cultivars and species and is useful for producing rice cultivars and novel types with the Rc-g trait.

Methods for introducing the allele into rice plants are described which entail crossing a rice plant which lacks the mutant allele with a rice plant that has the allele, selfing the resulting generations and then selecting the plants exhibiting a red pericarp.

In another aspect, a method for producing a hybrid rice seed is described which comprises crossing a first plant parent with a second plant parent and harvesting the resultant hybrid rice seed, wherein either one or both parents contain Rc-g, the mutant allele. The hybrid seeds, plant and parts thereof produced by such method are also described.

In another aspect, gene converted plants containing the mutant allele Rc-g are described. The desired transferred gene may preferably be a dominant or recessive allele. Preferably, the transferred gene will confer such trait as red pigmentation of the pericarp. The gene may be a naturally occurring rice gene, including a mutation or a transgene introduced through genetic engineering techniques.

In another aspect, regenerable cells may be for use in tissue culture of a rice plant containing Rc-g. The tissue culture will preferably be capable of regenerating plants having the physiological and morphological characteristics of the foregoing rice plant, and of regenerating plants having substantially the same genotype as the foregoing rice plant. Preferably, the regenerable cells in such tissue cultures will be embryos, protoplasts, meristematic cells, callus, pollen, leaves, anthers, pistils, roots, root tips, flowers, seeds, panicles or stems. Still further, rice plants may be regenerated from the tissue cultures of the invention.

Another aspect relates to any rice seed or plant having the mutant allele Rc-g.

A method is provided of producing a rice plant as well the rice plant itself, with a red-pigmented pericarp, wherein the method comprises transforming a rice plant with a transgene, wherein the transgene encodes a polypeptide having SEQ ID NO: 6.

A rice plant is described having the Rc-g allele, wherein at least 5% of grains resulting therefrom are red-pigmented.

Another aspect relates to a rice plant having a genotype having an allele selected from the group consisting of Rc-g/Rc-g and Rc-g/rc-g.

An isolated nucleic acid molecule is described having SEQ ID NO: 5 and wherein said nucleic acid encodes a polypeptide having SEQ ID NO: 6.

Another aspect relates to a polypeptide having amino acid deletions at positions 466, 467, 468, 469, and 470 and having amino acid substitutions at positions 463, 471, 472 and 474 of SEQ ID NO: 4.

Also described is an isolated nucleic acid molecule having a one base pair guanine deletion at position 1388 of SEQ ID NO: 3.

A method for testing red-pigmented grain for the Rc-g allele is described, comprising obtaining a polymerase chain reaction forward primer having SEQ ID NO: 10 and obtaining a polymerase chain reaction reverse primer having SEQ ID NO: 12 and obtaining a red grain sample and using said polymerase chain reaction forward and reverse primers for selectively amplifying the Rc-g allele in said grain sample.

Another aspect relates to a grain sample selected from the group consisting of a seed grain sample, a field grain sample, a harvested grain sample and a bagged grain sample.

In addition to the exemplary aspects and embodiments described above, further aspects and embodiments will become apparent by study of the following descriptions.

### DEFINITIONS

In the description and tables which follow, a number of terms are used. In order to provide a clear and consistent understanding of the specification and claims, including the scope to be given such terms, the following definitions are provided:

Alkali Spreading Value. Indicator of gelatinization temperature and an index that measures the extent of disintegration of milled rice kernel in contact with dilute alkali solution. Standard long grains have a 3 to 5 Alkali Spreading Value (intermediate gelatinization temperature).

Allele. An allele is any of one or more alternative forms of a gene which relate to one trait or characteristic. In a diploid cell or organism, the two alleles of a given gene occupy corresponding loci on a pair of homologous chromosomes.

Alter. The utilization of up-regulation, down-regulation, or gene silencing.

Apparent Amylose Percent. The most important grain characteristic that describes cooking behavior in each grain class, or type, i.e., long, medium and short grain. The percentage of the endosperm starch of milled rice that is amylose. Standard long grains contain 20 to 23% amylose. Rexmont-type long grains contain 24 to 25% amylose. Short and medium grains contain 16 to 19% amylose. Waxy rice contains 0% amylose. Amylose values will vary over environments.

Backcrossing. Backcrossing is a process in which a breeder successively crosses hybrid progeny back to one of the parents, for example, a first generation hybrid F₁ with one of the parental genotypes of the F₁ hybrid.

Bagged Grain Sample. A sample of rice seed that is bagged.

Cell. Cell as used herein includes a plant cell, whether isolated, in tissue culture or incorporated in a plant or plant part.

Cool Paste Viscosity. Viscosity measure of rice flour/water slurry after being heated to 95° C. and uniformly cooled to 50° C. (American Association of Cereal Chemist). Values less than 200 for cool paste indicate softer cooking types of rice.

Cotyledon. A cotyledon is a type of seed leaf. The cotyledon contains the food storage tissues of the seed.

Culm. The culm is the plant stem. It contains nodes and internodes. The internodes elongate as the plant matures pushing the younger leaves and ultimately pushing the inflorescence out through the leaf sheath of the flag leaf.

Days to 50% heading. Average number of days from planting to the day when 50% of all panicles are exerted at least partially through the leaf sheath. A measure of maturity.

Embryo. The embryo is the small plant contained within a mature seed.

Essentially all the physiological and morphological characteristics. A plant having essentially all the physiological and morphological characteristics means a plant having the physiological and morphological characteristics of the cultivar, except for the characteristics derived from one or more converted gene(s).

Field Grain Sample. A sample of rice seed that is or was collected in the field.

Flag Leaf. The flag leaf is the last leaf to emerge from the culm and is located just below the panicle. It consists of a sheath that is wrapped around the stem and a flat blade that extends from the sheath.

FNP. FNP as used herein means functional nucleotide polymorphism.

Gene Converted (Conversion). Gene converted (conversion) plant refers to plants which are developed by a plant breeding technique called backcrossing with selection wherein essentially all of the desired morphological and physiological characteristics of a variety are recovered in addition to the one or more genes transferred into the variety via the backcrossing technique, via genetic engineering or via mutation.

Gene Silencing. Gene silencing refers to the interruption or suppression of the expression of a gene at the level of transcription or translation.

Genotype. Genotype refers to the genetic constitution of a cell or organism.

Grain Length (L). The length of a rice grain is measured in millimeters.

Grain Width (W). The width of a rice grain is measured in millimeters.

Grain Yield. Grain yield is measured in pounds per acre and at 14.0% moisture. Grain yield of rice is determined by the number of panicles per unit area, the number of fertile florets per panicle, and grain weight per floret.

Harvest Moisture. Harvest moisture refers to the percent of moisture of the grain when harvested.

Harvested Grain Sample. A sample of rice seed collected from harvested rice.

Hot Past Viscosity. Viscosity measure of rice flour/water slurry after being heated to 95° C. Lower values indicate softer and more sticky cooking types of rice.

Hypocotyl. A hypocotyl is the portion of an embryo or seedling between the cotyledons and the root. Therefore, it can be considered a transition zone between shoot and root.

Leaf. The rice leaf consist of a sheath and a blade (lamina).The leaf sheath is an elongated part of the leaf rolled into a cylinder that encloses the developing new leaves and stem at later growth stages. The basal portion of the leaf sheath is attached to a nodal plate. The leaf blade is long and lanceolate with a midrib and has parallel veins on each side.

Leaf length. Leaf length is measured by measuring the longitudinal distance in centimeters of the mature rice leaf. Leaf blade length is the measured distance for the blade in centimeters from the attachment to the leaf sheath to the tip of the leaf.

Length/Width (L/W) Ratio. This ratio is determined by dividing the average grain length (L) by the average grain width (W).

Locus. A locus confers one or more traits such as, for example, male sterility, herbicide tolerance, insect resistance, disease resistance, and improved yield. The trait may be, for example, conferred by a naturally occurring gene introduced into the genome of the variety by backcrossing, a natural or induced mutation, or a transgene introduced through genetic transformation techniques. A locus may comprise one or more alleles integrated at a single chromosomal location.

Lodging Resistance (also called Straw Strength). Lodging is measured as a subjective rating and is a percentage of the plant stems leaning or fallen completely to the ground before harvest. It is a relative scale.

1000 Grain Wt. 1000 grain weight refers to the weight of 1000 rice grains as measured in grams. 1000 grain weight may be measured according to the form of rice including paddy (with hull), brown (without hull) or milled rice (without hull, germ and bran).

Panicle. Panicle refers to the inflorescence of the rice plant.

Panicle Length. Panicle length is the length of the rice panicle in centimeters from the base of the panicle to the tip of the panicle.

Pericarp. The pericarp comprises a thin cuticle covering the epidermis; several layers of partly crushed, “spongy” parenchyma; cross cells (with lignified cell walls) which are elongated in the transverse axis of the grain and tube cells.

Plant. As used herein, the term “plant” includes reference to an immature or mature whole plant, including a plant from which seed or grain or anthers have been removed. A seed or embryo that will produce the plant is also considered to be the plant.

Plant Height. Rice plant height is measured in centimeters from soil surface to the tip of the extended panicle at harvest.

Plant Parts. As used herein, the term “plant parts” (or a rice plant, or a part thereof) includes protoplasts, leaves, stems, roots, root tips, anthers, pistils, seed, grain, embryo, pollen, ovules, cotyledon, hypocotyl, panicles, glume, flower, shoot, tissue, petiole, cells, meristematic cells and the like.

Quantitative Trait Loci (QTL). Quantitative trait loci (QTL) refer to genetic loci that control to some degree numerically representable traits that are usually continuously distributed.

Red Grain Sample. A sample of rice seed that is red in color.

Regeneration. Regeneration refers to the development of a plant from tissue culture.

RVA. Rapid Visco Analyzer is a widely used laboratory instrument to examine paste viscosity, or thickening ability of milled rice during the cooking process.

Seed Grain Sample. A sample of rice seed.

Seedling emergence. Seedling emergence is the point at which the tip of the leaf of the growing rice seedling leaf emerges through the water in water seeded rice or the soil in direct seeded rice. This may be measured in days from planting to seedling emergence and will be the number or percentage of seedlings that have emerged.

Seedling Vigor. Seedling vigor refers to the ability of the seedling to emerge rapidly through the soil or water after planting. It is frequently measured by visual observation field test and assigned a relative score.

Stem internode length. Stem internode length is the longitudinal distance of an elongated internode (section of the stem between consecutive nodes) measured in centimeters.

Upper Internode Length. Upper internode length is the longitudinal distance in centimeters of the last fully elongated internode that is located just below the panicle.

### DETAILED DESCRIPTION OF THE INVENTION

Since the origins of agriculture humans have selected traits in plants that were useful in cropping systems. Propagation of specific variants, with repeated selection over time, imposed genetic bottlenecks that increased the uniformity of crops and fixed desirable traits in cultivated germplasm. Such traits in agricultural crops are often conferred by so-called ‘domestication genes’, which distinguish cultivated crops from their wild and frequently weedy progenitors. The vast majority of modern varieties of Oryza sativa L. have a white pericarp, which is conferred by the rc domestication pseudogene (Furukawa, T., et al., Plant J., 49:91-102 (2007) and Sweeney et al., Plant Cell 18:283-294 (2006)). White pericarp distinguishes cultivated rice from wild relative species, and also from ‘red rice’ which is a common weed of the same Oryza sativa L. species. These wild-types have the dominant red pericarp allele at the Rc locus.

Recently two groups independently cloned the Rc gene (Furukawa, T., et al., Plant J., 49:91-102 (2007) and Sweeney et al., Plant Cell 18:283-294 (2006)); and sequenced the wild-type allele (Rc), the domestication allele (rc), and a mutant allele (Rc-s; Sweeney et al., Plant Cell 18:283-294 (2006). Both groups demonstrated that rc was a null-allele, produced by a 14 base-pair deletion (in exon seven using the mRNA-based gene model of Furukawa, T., et al., Plant J., 49:91-102 (2007)), which caused a frame shift mutation and a premature stop codon. The rc mutation results in a loss of proanthocyanidin synthesis and corresponding loss of pigmentation of the pericarp (Furukawa, T., et al., Plant J., 49:91-102 (2007)).

It is speculated that selection for white rice grains was historically for sanitary purposes, where the trait permitted easy identification of impurities in stored grain. Post-harvest insect and rodent damage would be more easily detectable against a white rice background. The trait is also important to date for producing polished (milled) white rice. However, red pericarp can be desirable in specialty rices, and there are many good-quality, locally-adapted, red rice varieties grown throughout Asia. Unfortunately, these varieties are not adapted to the cultivation systems in the United States, and introgressing the Rc allele from non-adapted germplasm would require years of backcrossing and selection to produce useful varieties. Therefore, a source of red pericarp in adapted germplasm would be very valuable for specialty rice breeding in the United States.

Arkansas is a leading producer of rice in the United States, and all varieties in commercial production have a white pericarp (rc). Red rice is a common weed of Arkansas rice culture. Although it has the niche-desirable Rc allele, it does not possess cultivated plant characteristics, and is a noxious weed with no agronomic value. Red rice has medium grain length, is taller than conventional cultivars, shatters easily, and its seed can remain dormant in the soil between growing seasons. Red rice also outcrosses to cultivated rice at low frequency (Gealy, D R., et al., Weed Sci. 50:333-339 (2002)), causing great concern over genetic purity of cultivars and transfer of herbicide resistance to weed populations. Therefore, strong selection against red rice has been enforced in breeding programs, foundation seed, and on-farm operations, which limits the spread of weedy red rice and the monetary losses resulting from mixed-seed crops.

Recently typical long grains of the cultivar ‘Wells’ (U.S. Pat. No. 6,282,416) an extensively grown cultivar in Arkansas, were identified having red pericarp. ‘Wells’ is a high yielding, long-grain cultivar (Moldenhauer, K., et al., Crop Sci 47:442-443 (2007)). Plants grown from red seed were of cultivated idiotype purity which raised immediate concern regarding the genetic purity of ‘Wells’. Red rice is the only source of Rc in Arkansas rice production, and an outcross of ‘Wells’ to red rice would have undesired consequences.

The present invention is a new rice plant allele which arose by natural mutation within the rc pseudogene of the cultivar ‘Wells’ and is able to restore the reading frame of the gene, and reverts the bran layer pigmentation to red (Deren C. W., et al., Theor Appl Genet 117:575-580 (2008)). The present invention relates to a new and distinctive rice mutant allele designated “Rc-g”.

According to the invention, there is provided a novel rice mutant allele designated “Rc-g” that reverts white pericarp to red pericarp. This invention thus relates to the nucleotide sequence of the Rc-g allele, the amino acid sequence of the Rc-g allele, rice seeds containing the Rc-g allele, to rice plants containing the Rc-g allele and to methods for producing a rice plant by crossing a rice plant containing the Rc-g allele with itself or another rice line.

Thus, any such methods using rice containing the Rc-g allele are part of this invention: selling, backcrosses, hybrid production, crosses to populations, and the like. All plants produced using rice containing the Rc-g allele as a parent are within the scope of this invention.

Red rice contamination of cultivated seed lots is a common phenomenon in the southern United States, where red rice is a prevalent and problematic weed species. Red rice grain type is different from cultivated rice and can be excluded based upon grain shape and color. The identification of typical Wells long rice grains with red pericarp and cultivated idiotype in derived plants was alarming and posed a serious problem for certifying weed-free foundation seed. Since the probability of a mutation resulting in a dominant phenotype is low, an outcross was considered as the cause of red pericarp in red Wells. However, identical plant and grain types without the presence of red rice traits caused immediate doubt for the outcross hypothesis. Using the recently published DNA sequence for Rc (Sweeney et al. 2006) and markers for the gene, it was demonstrated that an unexpected mutation restored function to the rc pseudogene in the red Wells rice.

The presence of the rc functional nucleotide polymorphism (FNP) in red Wells eliminated an outcross from consideration as the source of red pericarp, since all wild-type red rices lack this 14 by deletion. Therefore, mutation either within rc or another gene in the same biochemical pathway were considered as the basis for the reversion phenotype. DNA sequence analysis revealed an unexpected 1 by deletion within the rc pseudogene in red Wells. This feature was surprising as it caused a reversion from domesticated-type rice pericarp color to wild-type rice pericarp color. The close proximity of two deletions within exon seven restored the reading frame of the gene, and unexpectedly resulted in an allele with little deviation from wild-type. Interpretation of the DNA sequences was convincing, but the perfect co-segregation of FNP and trait data provided sound evidence that a reversion to wild-type occurred via this mutation. The new allele was designated as Rc-g, symbolizing the guanine deletion as the allele's distinctive feature.

Rc-g is a naturally occurring mutation. At present the precise frequency of Rc-g occurrence in Wells is unknown, though the observed occurrence was equivalent to about a single plant in 56 tons of foundation seed. Low frequency of Rc-g is expected as a zero tolerance for red bran exists in the University of Arkansas foundation seed program, and is selected against to avoid red rice contamination of seed lots. In addition, Moldenhauer et al. (2007) reported off-type occurrence in Wells at a frequency of less than one in five thousand plants, and did not report red pericarp as an off-type for the cultivar.

Red rice is the most important weed problem of rice culture in the southern United States, causing significant economic losses in infested areas (summarized in Gealy et al. 2002). Since outcrossing of cultivated rice with red rice is known (Gealy et al. 2002), the ability to determine the source of red pericarp in red Wells was of paramount importance. Wells has been the leading inbred rice cultivar in Arkansas since 2002 (C. Wilson 2002), and genetic contamination of the cultivar would have had enormous economic impact. Identification of the Rc-g allele removes the assumption that cultivated rice-grain-types with red pericarp are always the result of outcrosses to red rice. (Similar phenotypic observations have been made in other cultivars, where typical long-grain-rice with red pericarp has been found). Use of the RID 12 (Sweeney et al. 2006) and the RCG-FNP markers will prove useful to identify the Rc, rc, and Rc-g alleles and to certify foundation seed lots.

The Red Wells mutant represents a novel germplasm resource for specialty rice breeding. Rc encodes a regulatory protein that enhances the accumulation of proanthocyanidins in the rice pericarp (Furukawa et al. 2007). The health beneficial properties of proanthocyanidin and related secondary metabolites are well known. Ling et al. (2001) demonstrated that red rice consumption reduced progression of atherosclerotic plaque development induced by dietary cholesterol (the area of aortic atherosclerotic plaques was 50% lower in rabbits fed red rice diets than those fed a white rice diet). The exploitation of Red Wells circumvents linkage drag from weedy traits (seed shattering and dormancy are tightly linked to Rc on chromosome 7; Ji et al. 2006), that occur when introgressing alleles like Rc from wild species. Red Wells has immediate utility as a specialty rice variety, and will prove useful as a source of the Rc-g allele (selectable with the RCG-FNP perfect marker) in an elite genetic background.

This invention is directed to any rice seed or plant containing the Rc-g allele. This invention also is directed to methods for producing a rice plant by crossing a first parent rice plant with a second parent rice plant wherein either the first or second parent rice plant is a rice plant containing the Rc-g allele. Further, both first and second parent rice plants can comprise the rice Rc-g allele. Still further, this invention also is directed to methods for producing a rice cultivar containing the Rc-g allele by crossing a rice cultivar containing the Rc-g allele with a second rice plant and growing the progeny seed, and repeating the crossing and growing steps with the rice cultivar containing the Rc-g allele from 0 to 7 times. Thus, any such methods using the rice Rc-g allele are part of this invention: selfing, backcrosses, hybrid production, crosses to populations, and the like. All plants produced using rice plants containing the Rc-g allele as parents are within the scope of this invention, including plants derived from rice Rc-g. Advantageously, the rice line is used in crosses with other, different, rice line to produce first generation (F₁) rice seeds and plants with superior characteristics.

It should be understood that rice plants containing the Rc-g allele can, through routine manipulation of cytoplasmic or other factors, be produced in a male-sterile form. Such embodiments are also contemplated within the scope of the present claims.

As used herein, the term plant includes plant cells, plant protoplasts, plant cell tissue cultures from which rice plants can be regenerated, plant calli, plant clumps and plant cells that are intact in plants or parts of plants, such as embryos, pollen, ovules, flowers, glumes, panicles, leaves, stems, roots, root tips, anthers, pistils and the like.

### EXAMPLES

The following examples are provided to further illustrate the present invention and are not intended to limit the invention beyond the limitations set forth in the appended claims.

### Example 1

### Plant Materials

The plant materials described herein were identified by the University of Arkansas, Rice Research and Extension Center foundation seed program, located in Stuttgart Arkansas. Rice kernels of typical long-grain-cultivated type were identified with red pericarp in 2005-produced seed of the cultivar ‘Wells’. These seed were clearly not of weedy red rice type, and were saved to determine if outcrossing was the source of red pericarp. Plants grown from the red-pericarp-rice were phenotypically identical to ‘Wells’, except for pericarp color, and the single plant selections were named ‘Red Wells’.

### Example 2

### Identification and Frequency of Red Wells

Using standard procedures required for certification of foundation seed (Arkansas State Plant Board 2002), approximately 150 red seeds were found in a 56 ton seed lot of the rice cultivar Wells. Single plant selections from white and red seeds were phenotypically identical for plant type, panicle morphology and grain type. Furthermore, 24 F₁s of reciprocal crosses made from the selected plants, and 96 derived F₂s (not shown), were all uniform for plant type. Quantitative measurements were also made for plant height, tiller number, days to heading, glabrous leaves (+/−), shattering and kernel dimensions (not shown). The only differences observed between Wells (n=18) and Red Wells (n=18) were in mean plant height (92±3 cm vs. 96±4 cm respectively) and kernel dimensions (7.6±0.2 mm vs. 7.1±0.2 mm for kernel length; and 2.0±0.1 mm vs. 1.9±0.2 mm for kernel width; Wells vs. Red Wells). The differences were statistically significant (p=0.05, df=34) for the single plant selections.

### Example 3

### Molecular Marker Analysis

Microsatellite markers (SSRs) were chosen based on availability, robust amplification, high polymorphism, and previous utility for ‘fingerprinting’ rice cultivars. Polymerase chain reaction (PCR) for SSRs was performed according to Gealy et al. (2002). The RID12 marker developed by Sweeney et al. (2006) was used to detect the Rc/rc functional nucleotide polymorphism (FNP). PCR reactions for RID12 were 20 μl in volume, used 50 ng template DNA, 1 pmol of each primer, 1 unit of Taq DNA polymerase (Promega, Madison, Wis.), 1.6 μl of 25 mM MgCl₂, 2 μl of 10×buffer, and 0.2 μl of 10 mM dNTPs. PCR was performed in an Eppendorf (Hamburg, Germany) Mastercycler, where reaction conditions were: 3 min at 94° C., followed by 35 cycles each of 1 min at 92° C., 1 min at 55° C., 1 min at 72° C., and a final cycle of 72° C. for five minutes.

### Example 4

### Red Wells did not Arise from an Outcross to Red Rice

Twenty two simple sequence repeat (SSR) markers, located on 10 of the 12 rice chromosomes, were used to search for non-Wells alleles in Red Wells (Table 1). All SSR markers produced identical allele sizes in Wells and Red Wells, and were different from Stuttgart straw hull (STG-S, except monomorphic marker RM420), the most common red rice type on the experiment station where Red Wells was identified. To assure that low marker resolution did not miss a localized introgression at the Rc locus, the RID12 marker, that distinguishes Rc/rc FNP was used to screen Wells, Red Wells, and three accessions of red rice. Wells and Red Wells had the rc FNP, which is 14 by smaller than the fragment amplified in Rc-allele-containing common red rice accessions (STG-S, RR8, and LA3). The presence of the rc FNP in Red Wells provides conclusive evidence that the dominant Rc phenotype was not the result of an outcross to an Rc-type red rice. Table 1 shows the SSR marker comparison between rice cultivar Wells and the version of Wells having the mutant Rc-g allele. In Table 1, column 1 shows the SSR marker, column 2 shows the chromosome location of the marker, column 3 shows the size of the allele in base pairs in Wells, column 4 shows the size of the allele in base pairs in Red Wells and column 5 shows the size of the allele in base pairs in STG-S, a type of weedy red rice.

TABLE 1
SSR marker comparison of ‘Wells’ and 'Red Wells'

| SSR Marker | | | | |
|---|---|---|---|---|
| (information can | Chromo- | | Allele size | Allele size |
| be found on the | some | Allele size | (bp) | (bp) |
| Gramene web site) | location | (bp) ‘Wells’ | ‘Red Wells’ | ‘STG-S’ |
| RM 5 | 1 | 114 | 114 | 109 |
| RM 22 | 3 | 191 | 191 | 194 |
| RM 124 | 4 | 271 | 271 | 267 |
| RM 125 | 7 | 126 | 126 | 123 |
| RM 154 | 2 | 179 | 179 | 165 |
| RM 174 | 2 | 221 | 221 | 206 |
| RM 190 | 6 | 113 | 113 | 107 |
| RM 210 | 8 | 151 | 151 | 153 |
| RM 214 | 7 | 149 | 149 | 112 |
| RM 219 | 9 | 190 | 190 | 220 |
| RM 224 | 11 | 139 | 139 | 128/154 |
| RM 225 | 6 | 142 | 142 | 130/142 |
| RM 231 | 3 | 180 | 180 | 184 |
| RM 232 | 3 | 158 | 158 | 148 |
| RM 234 | 7 | 135 | 135 | 153 |
| RM 248 | 7 | 84 | 84 | 91 |
| RM 345 | 6 | 163 | 163 | 169 |
| RM 408 | 8 | 127 | 127 | 125 |
| RM 420 | 7 | 186 | 186 | 186 |
| RM 481 | 7 | 217 | 217 | 176 |
| RM 484 | 10 | 298 | 298 | 293 |
| RM 490 | 1 | 97 | 97 | 93 |

As shown in Table 1, for this set of SSR markers, the alleles of Wells and Red Wells were identical and there were no alleles from the weedy red rice present in the Red Wells.

### Example 5

### RCG-FNP Distinguished the Rc-g and rc Alleles

A SNP-based PCR marker was developed to resolve the Rc-g/rc FNPs on standard agarose gels. FIG. 4 is a diagrammatic representation of primer binding positions. The RCG-FNP was then used to score 92 segregating F₂s derived from a cross between Wells and Red Wells, which had also been scored for pericarp color (F₃ seed, not shown). The critical values for chi-square are 3.84 for trait data (df=1, alpha=0.05), and 5.99 for marker data (df=2, alpha=0.05). The calculated chi-squares were 2.84 and 4.46 respectively, and failed to reject the null hypothesis for a single dominant gene. In all F₂ progeny perfect marker-trait co-segregation was observed, where Rc-g/Rc-g and Rc-g/rc genotypes had red seed and rc/rc genotypes were white seeded.

### Example 6

### DNA Sequence Analysis

A PCR-based approach was used to amplify the Rc locus, in overlapping fragments, for DNA sequencing. PCR primers were designed using PrimerSelect (DNASTAR, Madison, Wis.) based on the rc allele sequence of the rice cultivar ‘Jefferson’ (DQ204736). PCR conditions were nearly identical to those used for RID12, with the only difference being optimization of specific primer pair annealing temperatures. Direct sequencing of PCR products was performed by the Genomics Core Facility at the Dale Bumpers National Rice Research Center. DNA sequence assembly and alignment was performed with SeqMan II (DNASTAR, Madison, Wis.).

### Example 7

### Rc-g is a Novel, Dominant Allele that Arose by Natural Mutation in ‘Red Wells’

DNA sequence alignments of Wells and Red Wells within the exon seven region of the O. rufipogon (NCBI Accession No. DQ204737) Rc gene sequence (FIG. 3), revealed a 1 by deletion (guanine) in Red Wells at position 1388. This feature is 20 by upstream of the rc FNP and lies within the RID12 amplicon. Therefore, both Wells and Red Wells have the rc FNP (14 by deletion), but Red Wells has an additional 1 by deletion resulting in a RID12 fragment that is 15 by shorter than obtained from a Rc allele. The 1 by deletion was the only sequence polymorphism observed between Wells and Red Wells. The rc coding sequence in Wells was also identical to the published sequence for the rice cultivar Jefferson (NCBI Accession No. DQ204736).

The close proximity of two deletions within exon seven, upstream of the functional domain, restored the reading frame of the rc pseudogene to a functional allele (Rc-g) in Red Wells. Putative amino acid (aa) sequence alignments based on the O. rufipogon gene model (Sweeney et al. 2006) show the premature stop codon in Wells (rc) at aa position 474 (FIG. 3) that results from the 14 by FNP. The Rc-g allele encodes a putative full-length polypeptide, with a five aa deletion (positions 466-470) and four aa substitutions (positions 463, 471, 472, and 474) relative to the Rc protein sequence. Perfect alignment exists between Rc and Rc-g aa sequences before aa-463 and following aa-474 to the end of the protein (668 aa total for Rc; 663 aa for Rc-g).

### Example 8

### RCG-FNP Marker Development for Use in Screening

Two single nucleotide polymorphism (SNP)-based PCR primer pairs were designed to selectively amplify the Rc-g and rc alleles. Forward primers were designed to terminate on a single selective base, at position 1388 of the O. rufipogon Rc coding sequence (DQ204737). pSNP-red (5′-AGAAACACCTGAATCAATGGC-3′) SEQ ID NO: 10 and pRedReverse (5′-GAGCTCTTGTATGCGGTTCCTTAG-3′) SEQ ID NO: 12 produce an 889 by fragment from the Rc-g allele. pSNP-white (5′-AGAAACACCTGAATCAAGTGG-3′) SEQ ID NO: 7 and pWhiteReverse (5′-GGATACGGGTAGGATTCACTTCTG-3′) SEQ ID NO: 13 produce a 668 by fragment from the rc allele. Since individual primer pairs were dominant markers, both sets were used individually (on all samples) and pooled after amplification, to produce an essentially co-dominant FNP fingerprint. Different reverse primers were used to produce amplicons of significantly different size that could be resolved on agarose gels. The pSNP-white primer will also amplify the Rc allele (red), but the fragment will be 14 by larger than the rc fragment, as these primers also span the Rc/rc FNP. Therefore, the RCG-FNP marker, a combination of two dominant PCR products, can be used to score all three (Rc, Rc-g and rc) alleles simultaneously. PCR reactions for the RCG-FNP were 20 μl in volume, used 100 ng template DNA, 0.5 pmol of each primer, 1 unit of Taq DNA polymerase (Promega, Madison, Wis.), 1.4 ml of 25 mM MgCl₂, 2 μl of 10×buffer, and 0.2 μl of 10 mM dNTPs. PCR was performed in an Eppendorf (Hamburg, Germany) Mastercycler and reaction conditions were: 94° C. for 3 min, followed by 35 cycles of 92° C. for 2 min, 45 seconds at 60° C. (pSNP-red) or 62° C. (pSNP-white), 72° C. for 1 min 30 sec, and a final cycle of 72° C. for 10 minutes. All samples were tested in triplicate and analyzed on 1.5% agarose (Bio-Rad, Hercules, Calif.) gels.

### Example 9

### F2 Mapping Population

Reciprocal crosses from single plant selections of Wells and Red Wells were made in the spring of 2006 and F₁ plants grown in the summer of the same year. F₂ seed was collected from a single F₁ (Wells×Red Wells), and 92 Fₑs were grown in the greenhouse during the winter of 2006-2007. Leaf tissue was harvested for DNA extraction and molecular marker analysis at the seedling 4 leaf stage. A single panicle from each F₂ was collected at maturity and de-hulled (palea and lemma removed) to score pericarp color.

### FURTHER EMBODIMENTS OF THE INVENTION

With the advent of molecular biological techniques that have allowed the isolation and characterization of genes that encode specific protein products, scientists in the field of plant biology developed a strong interest in engineering the genome of plants to contain and express foreign genes, or additional, or modified versions of native, or endogenous, genes (perhaps driven by different promoters) in order to alter the traits of a plant in a specific manner. Such foreign additional and/or modified genes are referred to herein collectively as “transgenes”. Over the last fifteen to twenty years, several methods for producing transgenic plants have been developed, and the present invention, in particular embodiments, also relates to transformed versions of the claimed cultivar.

Culture for expressing desired structural genes and cultured cells are known in the art. Also as known in the art, rice is transformable and regenerable such that whole plants containing and expressing desired genes under regulatory control may be obtained. General descriptions of plant expression vectors and reporter genes and transformation protocols can be found in Gruber, et al., “Vectors for Plant Transformation”, in Methods in Plant Molecular Biology & Biotechnology in Glich, et al., (Eds. pp. 89-119, CRC Press, 1993). Moreover GUS expression vectors and GUS gene cassettes are available from Clone Tech Laboratories, Inc., Palo Alto, Calif. while luciferase expression vectors and luciferase gene cassettes are available from Pro Mega Corp. (Madison, Wis.). General methods of culturing plant tissues are provided for example by Maki, et al., “Procedures for Introducing Foreign DNA into Plants” in Methods in Plant Molecular Biology & Biotechnology, Glich, et al., (Eds. pp. 67-88 CRC Press, 1993); and by Phillips, et al., “Cell-Tissue Culture and In-Vitro Manipulation” in Corn & Corn Improvement, 3rd Edition; Sprague, et al., (Eds. pp. 345-387 American Society of Agronomy Inc., 1988). Methods of introducing expression vectors into plant tissue include the direct infection or co-cultivation of plant cells with Agrobacterium tumefaciens, described for example by Horsch et al., Science, 227:1229 (1985). Descriptions of Agrobacterium vector systems and methods for Agrobacterium-mediated gene transfer are provided by Gruber, et al., supra.

Useful methods include but are not limited to expression vectors introduced into plant tissues using a direct gene transfer method such as microprojectile-mediated delivery, DNA injection, electroporation and the like. More preferably expression vectors are introduced into plant tissues using a microprojectile media delivery system with a biolistic device or using Agrobacterium-mediated transformation. Transformant plants obtained with the protoplasm of the invention are intended to be within the scope of this invention.

Plant transformation involves the construction of an expression vector which will function in plant cells. Such a vector comprises DNA comprising a gene under control of or operatively linked to a regulatory element (for example, a promoter). The expression vector may contain one or more such operably linked gene/regulatory element combinations. The vector(s) may be in the form of a plasmid, and can be used alone or in combination with other plasmids, to provide transformed rice plants, using transformation methods as described below to incorporate transgenes into the genetic material of the rice plant(s).

### Expression Vectors for Transformation: Marker Genes

Expression vectors include at least one genetic marker, operably linked to a regulatory element (a promoter, for example) that allows transformed cells containing the marker to be either recovered by negative selection, i.e., inhibiting growth of cells that do not contain the selectable marker gene, or by positive selection, i.e., screening for the product encoded by the genetic marker. Many commonly used selectable marker genes for plant transformation are well known in the transformation arts, and include, for example, genes that code for enzymes that metabolically detoxify a selective chemical agent which may be an antibiotic or an herbicide, or genes that encode an altered target which is insensitive to the inhibitor. A few positive selection methods are also known in the art.

One commonly used selectable marker gene for plant transformation is the neomycin phosphotransferase II (nptII) gene, isolated from transposon Tn5, which when placed under the control of plant regulatory signals confers resistance to kanamycin. Fraley et al., Proc. Natl. Acad. Sci. U.S.A., 80:4803 (1983). Another commonly used selectable marker gene is the hygromycin phosphotransferase gene which confers resistance to the antibiotic hygromycin. Vanden Elzen et al., Plant Mol. Biol., 5:299 (1985).

Additional selectable marker genes of bacterial origin that confer resistance to antibiotics include gentamycin acetyl transferase, streptomycin phosphotransferase, and aminoglycoside-3′-adenyl transferase, the bleomycin resistance determinant. Hayford et al., Plant Physiol. 86:1216 (1988); Jones et al., Mol. Gen. Genet., 210:86 (1987); Svab et al., Plant Mol. Biol. 14:197 (1990); Hille et al., Plant Mol. Biol. 7:171 (1986). Other selectable marker genes confer resistance to herbicides such as glyphosate, glufosinate or bromoxynil. Comai et al., Nature 317:741-744 (1985), Gordon-Kamm et al., Plant Cell 2:603-618 (1990) and Stalker et al., Science 242:419-423 (1988).

Selectable marker genes for plant transformation not of bacterial origin include, for example, mouse dihydrofolate reductase, plant 5-enolpyruvylshikimate-3-phosphate synthase and plant acetolactate synthase. Eichholtz et al., Somatic Cell Mol. Genet. 13:67 (1987), Shah et al., Science 233:478 (1986), Charest et al., Plant Cell Rep. 8:643 (1990).

Another class of marker genes for plant transformation requires screening of presumptively transformed plant cells rather than direct genetic selection of transformed cells for resistance to a toxic substance such as an antibiotic. These genes are particularly useful to quantify or visualize the spatial pattern of expression of a gene in specific tissues and are frequently referred to as reporter genes because they can be fused to a gene or gene regulatory sequence for the investigation of gene expression. Commonly used genes for screening presumptively transformed cells include β-glucuronidase (GUS), β-galactosidase, luciferase and chloramphenicol acetyltransferase. Jefferson, R. A., Plant Mol. Biol. Rep. 5:387 (1987), Teeri et al., EMBO J. 8:343 (1989), Koncz et al., Proc. Natl. Acad. Sci U.S.A. 84:131 (1987), DeBlock et al., EMBO J. 3:1681 (1984). Another approach to the identification of relatively rare transformation events has been use of a gene that encodes a dominant constitutive regulator of the Zea mays anthocyanin pigmentation pathway. Ludwig et al., Science 247:449 (1990).

In vivo methods for visualizing GUS activity that do not require destruction of plant tissue are available. Molecular Probes publication 2908, IMAGENE GREEN, p. 1-4 (1993) and Naleway et al., J. Cell Biol. 115:151 a (1991). However, these in vivo methods for visualizing GUS activity have not proven useful for recovery of transformed cells because of low sensitivity, high fluorescent backgrounds and limitations associated with the use of luciferase genes as selectable markers.

More recently, a gene encoding Green Fluorescent Protein (GFP) has been utilized as a marker for gene expression in prokaryotic and eukaryotic cells. Chalfie et al., Science 263:802 (1994). GFP and mutants of GFP may be used as screenable markers.

### Expression Vectors for Transformation: Promoters

Genes included in expression vectors must be driven by nucleotide sequence comprising a regulatory element, for example, a promoter. Several types of promoters are now well known in the transformation arts, as are other regulatory elements that can be used alone or in combination with promoters.

As used herein, “promoter” includes reference to a region of DNA upstream from the start of transcription and involved in recognition and binding of RNA polymerase and other proteins to initiate transcription. A “plant promoter” is a promoter capable of initiating transcription in plant cells. Examples of promoters under developmental control include promoters that preferentially initiate transcription in certain tissues, such as leaves, roots, seeds, fibers, xylem vessels, tracheids, or sclerenchyma. Such promoters are referred to as “tissue-preferred”. Promoters which initiate transcription only in certain tissue are referred to as “tissue-specific”. A “cell type” specific promoter primarily drives expression in certain cell types in one or more organs, for example, vascular cells in roots or leaves. An “inducible” promoter is a promoter which is under environmental control. Examples of environmental conditions that may effect transcription by inducible promoters include anaerobic conditions or the presence of light. Tissue-specific, tissue-preferred, cell type specific, and inducible promoters constitute the class of “non-constitutive” promoters. A “constitutive” promoter is a promoter which is active under most environmental conditions.

A. Inducible Promoters—An inducible promoter is operably linked to a gene for expression in rice. Optionally, the inducible promoter is operably linked to a nucleotide sequence encoding a signal sequence which is operably linked to a gene for expression in rice. With an inducible promoter the rate of transcription increases in response to an inducing agent.

Any inducible promoter can be used in the instant invention. See Ward et al., Plant Mol. Biol. 22:361-366 (1993). Exemplary inducible promoters include, but are not limited to, that from the ACEI system which responds to copper (Meft et al., PNAS 90:4567-4571 (1993)); In2 gene from maize which responds to benzenesulfonamide herbicide safeners (Hershey et al., Mol. Gen Genetics 227:229-237 (1991) and Gatz et al., Mol. Gen. Genetics 243:32-38 (1994)) or Tet repressor from Tn10 (Gatz et al., Mol. Gen. Genetics 227:229-237 (1991)). A particularly preferred inducible promoter is a promoter that responds to an inducing agent to which plants do not normally respond. An exemplary inducible promoter is the inducible promoter from a steroid hormone gene, the transcriptional activity of which is induced by a glucocorticosteroid hormone (Schena et al., Proc. Natl. Acad. Sci. U.S.A. 88:0421 (1991)).

B. Constitutive Promoters—A constitutive promoter is operably linked to a gene for expression in rice or the constitutive promoter is operably linked to a nucleotide sequence encoding a signal sequence which is operably linked to a gene for expression in rice.

Many different constitutive promoters can be utilized in the instant invention. Exemplary constitutive promoters include, but are not limited to, the promoters from plant viruses such as the 35S promoter from CaMV (Odell et al., Nature 313:810-812 (1985)) and the promoters from such genes as rice actin (McElroy et al., Plant Cell 2:163-171 (1990)); ubiquitin (Christensen et al., Plant Mol. Biol. 12:619-632 (1989) and Christensen et al., Plant Mol. Biol. 18:675-689 (1992)); pEMU (Last et al., Theor. Appl. Genet. 81:581-588 (1991)); MAS (Velten et al., EMBO J. 3:2723-2730 (1984)) and maize H3 histone (Lepetit et al., Mol. Gen. Genetics 231:276-285 (1992) and Atanassova et al., Plant Journal 2 (3): 291-300 (1992)).

The ALS promoter, Xbal/Ncol fragment 5′ to the Brassica napus ALS3 structural gene (or a nucleotide sequence similar to said Xbal/Ncol fragment), represents a particularly useful constitutive promoter. See PCT application WO96/30530.

C. Tissue-specific or Tissue-preferred Promoters—A tissue-specific promoter is operably linked to a gene for expression in rice. Optionally, the tissue-specific promoter is operably linked to a nucleotide sequence encoding a signal sequence which is operably linked to a gene for expression in rice. Plants transformed with a gene of interest operably linked to a tissue-specific promoter produce the protein product of the transgene exclusively, or preferentially, in a specific tissue.

Any tissue-specific or tissue-preferred promoter can be utilized in the instant invention. Exemplary tissue-specific or tissue-preferred promoters include, but are not limited to, a root-preferred promoter, such as that from the phaseolin gene (Murai et al., Science 23:476-482 (1983) and Sengupta-Gopalan et al., Proc. Natl. Acad. Sci. U.S.A. 82:3320-3324 (1985)); a leaf-specific and light-induced promoter such as that from cab or rubisco (Simpson et al., EMBO J. 4(11):2723-2729 (1985) and Timko et al., Nature 318:579-582 (1985)); an anther-specific promoter such as that from LAT52 (Twell et al., Mol. Gen. Genetics 217:240-245 (1989)); a pollen-specific promoter such as that from Zm13 (Guerrero et al., Mol. Gen. Genetics 244:161-168 (1993)) or a microspore-preferred promoter such as that from apg (Twell et al., Sex. Plant Reprod. 6:217-224 (1993)).

### Signal Sequences for Targeting Proteins to Subcellular Compartments

Transport of protein produced by transgenes to a subcellular compartment such as the chloroplast, vacuole, peroxisome, glyoxysome, cell wall or mitochondrion or for secretion into the apoplast, is accomplished by means of operably linking the nucleotide sequence encoding a signal sequence to the 5′ and/or 3′ region of a gene encoding the protein of interest. Targeting sequences at the 5′ and/or 3′ end of the structural gene may determine, during protein synthesis and processing, where the encoded protein is ultimately compartmentalized.

The presence of a signal sequence directs a polypeptide to either an intracellular organelle or subcellular compartment or for secretion to the apoplast. Many signal sequences are known in the art. See, for example Becker et al., Plant Mol. Biol. 20:49 (1992); Knox, C., et al., Plant Mol. Biol. 9:3-17 (1987); Lerner et al., Plant Physiol. 91:124-129 (1989); Fontes et al., Plant Cell 3:483-496 (1991); Matsuoka et al., Proc. Natl. Acad. Sci. 88:834 (1991); Gould et al., J. Cell. Biol. 108:1657 (1989); Creissen et al., pu Plant J. 2:129 (1991); Kalderon, et al., Cell 39:499-509 (1984); Steifel, et al., Plant Cell 2:785-793 (1990).

### Foreign Protein Genes and Agronomic Genes

With transgenic plants according to the present invention, a foreign protein can be produced in commercial quantities. Thus, techniques for the selection and propagation of transformed plants, which are well understood in the art, yield a plurality of transgenic plants which are harvested in a conventional manner, and a foreign protein then can be extracted from a tissue of interest or from total biomass. Protein extraction from plant biomass can be accomplished by known methods which are discussed, for example, by Heney and Orr, Anal. Biochem. 114:92-6 (1981).

According to a preferred embodiment, the transgenic plant provided for commercial production of foreign protein is rice. In another preferred embodiment, the biomass of interest is seed. For the relatively small number of transgenic plants that show higher levels of expression, a genetic map can be generated, primarily via conventional RFLP, PCR and SSR analysis, which identifies the approximate chromosomal location of the integrated DNA molecule. For exemplary methodologies in this regard, see Glick and Thompson, Methods in Plant Molecular Biology and Biotechnology. CRC Press, Boca Raton. 269:284 (1993). Map information concerning chromosomal location is useful for proprietary protection of a subject transgenic plant. If unauthorized propagation is undertaken and crosses made with other germplasm, the map of the integration region can be compared to similar maps for suspect plants, to determine if the latter have a common parentage with the subject plant. Map comparisons would involve hybridizations, RFLP, PCR, SSR and sequencing, all of which are conventional techniques.

Likewise, by means of the present invention, agronomic genes can be expressed in transformed plants. More particularly, plants can be genetically engineered to express various phenotypes of agronomic interest. Exemplary genes implicated in this regard include, but are not limited to, those categorized below:
1. Genes That Confer Resistance to Pests or Disease and That Encode:

A. Plant disease resistance genes. Plant defenses are often activated by specific interaction between the product of a disease resistance gene (R) in the plant and the product of a corresponding avirulence (Avr) gene in the pathogen. A plant cultivar can be transformed with cloned resistance gene to engineer plants that are resistant to specific pathogen strains. See, for example Jones et al., Science 266:789 (1994) (cloning of the tomato Cf-9 gene for resistance to Cladosporium fulvum); Martin et al., Science 262:1432 (1993) (tomato Pto gene for resistance to Pseudomonas syringae pv. tomato encodes a protein kinase); Mindrinos et al., Cell 78:1089 (1994) (Arabidopsis RSP2 gene for resistance to Pseudomonas syringae).

B. A Bacillus thuringiensis protein, a derivative thereof or a synthetic polypeptide modeled thereon. See, for example, Geiser et al., Gene 48:109 (1986), who disclose the cloning and nucleotide sequence of a Bt δ-endotoxin gene. Moreover, DNA molecules encoding δ-endotoxin genes can be purchased from American Type Culture Collection, Manassas, Va., for example, under ATCC Accession Nos. 40098, 67136, 31995 and 31998.

C. A lectin. See, for example, the disclosure by Van Damme et al., Plant Molec. Biol. 24:25 (1994), who disclose the nucleotide sequences of several Clivia miniata mannose-binding lectin genes.

D. A vitamin-binding protein such as avidin. See PCT application US93/06487. The application teaches the use of avidin and avidin homologues as larvicides against insect pests.

E. An enzyme inhibitor, for example, a protease or proteinase inhibitor or an amylase inhibitor. See, for example, Abe et al., J. Biol. Chem. 262:16793 (1987) (nucleotide sequence of rice cysteine proteinase inhibitor), Huub et al., Plant Molec. Biol. 21:985 (1993) (nucleotide sequence of cDNA encoding tobacco proteinase inhibitor I), Sumitani et al., Biosci. Biotech. Biochem. 57:1243 (1993) (nucleotide sequence of Streptomyces nitrosporeus α-amylase inhibitor).

F. An insect-specific hormone or pheromone such as an ecdysteroid and juvenile hormone, a variant thereof, a mimetic based thereon, or an antagonist or agonist thereof. See, for example, the disclosure by Hammock et al., Nature 344:458 (1990), of baculovirus expression of cloned juvenile hormone esterase, an inactivator of juvenile hormone.

G. An insect-specific peptide or neuropeptide which, upon expression, disrupts the physiology of the affected pest. For example, see the disclosures of Regan, J. Biol. Chem. 269:9 (1994) (expression cloning yields DNA coding for insect diuretic hormone receptor), and Pratt et al., Biochem. Biophys. Res. Comm. 163:1243 (1989) (an allostatin is identified in Diploptera puntata). See also U.S. Pat. No. 5,266,317 to Tomalski et al., who disclose genes encoding insect-specific, paralytic neurotoxins.

H. An insect-specific venom produced in nature by a snake, a wasp, etc. For example, see Pang et al., Gene 116:165 (1992), for disclosure of heterologous expression in plants of a gene coding for a scorpion insectotoxic peptide.

I. An enzyme responsible for a hyper-accumulation of a monoterpene, a sesquiterpene, a steroid, a hydroxamic acid, a phenylpropanoid derivative or another non-protein molecule with insecticidal activity.

J. An enzyme involved in the modification, including the post-translational modification, of a biologically active molecule; for example, a glycolytic enzyme, a proteolytic enzyme, a lipolytic enzyme, a nuclease, a cyclase, a transaminase, an esterase, a hydrolase, a phosphatase, a kinase, a phosphorylase, a polymerase, an elastase, a chitinase and a glucanase, whether natural or synthetic. See PCT application WO 93/02197 in the name of Scott et al., which discloses the nucleotide sequence of a callase gene. DNA molecules which contain chitinase-encoding sequences can be obtained, for example, from the ATCC under Accession Nos. 39637 and 67152. See also Kramer et al., Insect Biochem. Molec. Biol. 23:691 (1993), who teach the nucleotide sequence of a cDNA encoding tobacco hornworm chitinase, and Kawalleck et al., Plant Molec. Biol. 21:673 (1993), who provide the nucleotide sequence of the parsley ubi4-2 polyubiquitin gene.

K. A molecule that stimulates signal transduction. For example, see the disclosure by Botella et al., Plant Molec. Biol. 24:757 (1994), of nucleotide sequences for mung bean calmodulin cDNA clones, and Griess et al., Plant Physiol. 104:1467 (1994), who provide the nucleotide sequence of a maize calmodulin cDNA clone.

L. A hydrophobic moment peptide. See PCT application WO95/16776 (disclosure of peptide derivatives of Tachyplesin which inhibit fungal plant pathogens) and PCT application WO95/18855 (teaches synthetic antimicrobial peptides that confer disease resistance), the respective contents of which are hereby incorporated by reference.

M. A membrane permease, a channel former or a channel blocker. For example, see the disclosure of Jaynes et al., Plant Sci 89:43 (1993), of heterologous expression of a cecropin-13, lytic peptide analog to render transgenic tobacco plants resistant to Pseudomonas solanacearum.

N. A viral-invasive protein or a complex toxin derived therefrom. For example, the accumulation of viral coat proteins in transformed plant cells imparts resistance to viral infection and/or disease development effected by the virus from which the coat protein gene is derived, as well as by related viruses. See Beachy et al., Ann. Rev. Phytopathol. 28:451 (1990). Coat protein-mediated resistance has been conferred upon transformed plants against alfalfa mosaic virus, cucumber mosaic virus, tobacco streak virus, potato virus X, potato virus Y, tobacco etch virus, tobacco rattle virus and tobacco mosaic virus. Id.

O. An insect-specific antibody or an immunotoxin derived therefrom. Thus, an antibody targeted to a critical metabolic function in the insect gut would inactivate an affected enzyme, killing the insect. Cf. Taylor et al., Abstract #497, Seventh Int'l Symposium on Molecular Plant-Microbe Interactions (Edinburgh, Scotland) (1994) (enzymatic inactivation in transgenic tobacco via production of single-chain antibody fragments).

P. A virus-specific antibody. See, for example, Tavladoraki et al., Nature 366:469 (1993), who show that transgenic plants expressing recombinant antibody genes are protected from virus attack.

Q. A developmental-arrestive protein produced in nature by a pathogen or a parasite. Thus, fungal endo-α-1,4-D-polygalacturonases facilitate fungal colonization and plant nutrient release by solubilizing plant cell wall homo-α-1,4-D-galacturonase. See Lamb et al., Bio/Technology 10:1436 (1992). The cloning and characterization of a gene which encodes a bean endopolygalacturonase-inhibiting protein is described by Toubart et al., Plant J. 2:367 (1992).

R. A developmental-arrestive protein produced in nature by a plant. For example, Logemann et al., Bio/Technology 10:305 (1992), have shown that transgenic plants expressing the barley ribosome-inactivating gene have an increased resistance to fungal disease.
2. Genes That Confer Resistance to an Herbicide, for Example:

A. An herbicide that inhibits the growing point or meristem, such as an imidazolinone or a sulfonylurea. Exemplary genes in this category code for mutant ALS and AHAS enzyme as described, for example, by Lee et al., EMBO J. 7:1241 (1988), and Miki et al., Theor. AppL Genet. 80:449 (1990), respectively.

B. Glyphosate (resistance conferred by mutant 5-enolpyruvylshikimate-3-phosphate synthase (EPSP) and aroA genes, respectively) and other phosphono compounds such as glufosinate (phosphinothricin acetyl transferase (PAT) and Streptomyces hygroscopicus PAT, bar, genes), and pyridinoxy or phenoxy propionic acids and cyclohexones (ACCase inhibitor-encoding genes). See, for example, U.S. Pat. No. 4,940,835 to Shah, et al., which discloses the nucleotide sequence of a form of EPSP which can confer glyphosate resistance. A DNA molecule encoding a mutant aroA gene can be obtained under ATCC accession number 39256, and the nucleotide sequence of the mutant gene is disclosed in U.S. Pat. No. 4,769,061 to Comai. European patent application No. 0 333 033 to Kumada et al., and U.S. Pat. No. 4,975,374 to Goodman et al., disclose nucleotide sequences of glutamine synthetase genes which confer resistance to herbicides such as L-phosphinothricin. The nucleotide sequence of a PAT gene is provided in European application No. 0 242 246 to Leemans et al. DeGreef et al., Bio/Technology 7:61 (1989), describe the production of transgenic plants that express chimeric bar genes coding for PAT activity. Exemplary of genes conferring resistance to phenoxy propionic acids and cyclohexones, such as sethoxydim and haloxyfop are the Accl-S1, Accl-S2 and Accl-S3 genes described by Marshall et al., Theor. Appl. Genet. 83:435 (1992).

C. An herbicide that inhibits photosynthesis, such as a triazine (psbA and gs+ genes) or a benzonitrile (nitrilase gene). Przibilla et al., Plant Cell 3:169 (1991), describe the transformation of Chlamydomonas with plasmids encoding mutant psbA genes. Nucleotide sequences for nitrilase genes are disclosed in U.S. Pat. No. 4,810,648 to Stalker, and DNA molecules containing these genes are available under ATCC Accession Nos. 53435, 67441, and 67442. Cloning and expression of DNA coding for a glutathione S-transferase is described by Hayes et al., Biochem. J. 285:173 (1992).
3. Genes That Confer or Contribute to a Value-Added Trait, Such as:

A. Modified fatty acid metabolism, for example, by transforming a plant with an antisense gene of stearyl-ACP desaturase to increase stearic acid content of the plant. See Knultzon et al., Proc. Natl. Acad. Sci. U.S.A. 89:2624 (1992).

B. Decreased phytate content, 1) Introduction of a phytase-encoding gene would enhance breakdown of phytate, adding more free phosphate to the transformed plant. For example, see Van Hartingsveldt et al., Gene 127:87 (1993), for a disclosure of the nucleotide sequence of an Aspergillus niger phytase gene; 2) A gene could be introduced that reduced phytate content. In maize, this, for example, could be accomplished, by cloning and then reintroducing DNA associated with the single allele which is responsible for maize mutants characterized by low levels of phytic acid. See Raboy et al., Maydica 35:383 (1990).

C. Modified carbohydrate composition effected, for example, by transforming plants with a gene coding for an enzyme that alters the branching pattern of starch. See Shiroza et al., J. Bacteol. 170:810 (1988) (nucleotide sequence of Streptococcus mutants fructosyltransferase gene), Steinmetz et al., Mol. Gen. Genet. 20:220 (1985) (nucleotide sequence of Bacillus subtilis levansucrase gene), Pen et al., Bio/Technology 10:292 (1992) (production of transgenic plants that express Bacillus lichenifonnis α-amylase), Elliot et al., Plant Molec. Biol. 21:515 (1993) (nucleotide sequences of tomato invertase genes), Søgaard et al., J. Biol. Chem. 268:22480 (1993) (site-directed mutagenesis of barley α-amylase gene), and Fisher et al., Plant Physiol. 102:1045 (1993) (maize endosperm starch branching enzyme II).

### Methods for Transformation

Numerous methods for plant transformation have been developed, including biological and physical, plant transformation protocols. See, for example, Miki et al., “Procedures for Introducing Foreign DNA into Plants” in Methods in Plant Molecular Biology and Biotechnology, Glick B. R. and Thompson, J. E. Eds. (CRC Press, Inc., Boca Raton, 1993) pages 67-88. In addition, expression vectors and in vitro culture methods for plant cell or tissue transformation and regeneration of plants are available. See, for example, Gruber et al., “Vectors for Plant Transformation” in Methods in Plant Molecular Biology and Biotechnology, Glick B. R. and Thompson, J. E. Eds. (CRC Press, Inc., Boca Raton, 1993) pages 89-119.

A. Agrobacterium-mediated Transformation—One method for introducing an expression vector into plants is based on the natural transformation system of Agrobacterium. See, for example, Horsch et al., Science 227:1229 (1985). A. tumefaciens and A. rhizogenes are plant pathogenic soil bacteria which genetically transform plant cells. The Ti and Ri plasmids of A. tumefaciens and A. rhizogenes, respectively, carry genes responsible for genetic transformation of the plant. See, for example, Kado, C. I., Crit. Rev. Plant Sci. 10:1 (1991). Descriptions of Agrobacterium vector systems and methods for Agrobacterium-mediated gene transfer are provided by Gruber et al., supra, Miki et al., supra, and Moloney et al., Plant Cell Reports 8:238 (1989). See also, U.S. Pat. No. 5,591,616 issued Jan. 7, 1997.

B. Direct Gene Transfer—Despite the fact the host range for Agrobacterium-mediated transformation is broad, some major cereal crop species and gymnosperms have generally been recalcitrant to this mode of gene transfer, even though some success has recently been achieved in rice and corn. Hiei et al., The Plant Journal 6:271-282 (1994) and U.S. Pat. No. 5,591,616 issued Jan. 7, 1997. Several methods of plant transformation, collectively referred to as direct gene transfer, have been developed as an alternative to Agrobacterium-mediated transformation.

A generally applicable method of plant transformation is microprojectile-mediated transformation wherein DNA is carried on the surface of microprojectiles measuring 1 to 4 um. The expression vector is introduced into plant tissues with a biolistic device that accelerates the microprojectiles to speeds of 300 to 600 m/s which is sufficient to penetrate plant cell walls and membranes. Sanford et al., Part. Sci. Technol. 5:27 (1987), Sanford, J. C., Trends Biotech. 6:299 (1988), Klein et al., Bio/Technology 6:559-563 (1988), Sanford, J. C., Physiol Plant 7:206 (1990), Klein et al., Bio/Technology 10:268 (1992). In corn, several target tissues can be bombarded with DNA-coated microprojectiles in order to produce transgenic plants, including, for example, callus (Type I or Type II), immature embryos, and meristematic tissue.

Another method for physical delivery of DNA to plants is sonication of target cells. Zhang et al., Bio/Technology 9:996 (1991). Additionally, liposome and spheroplast fusion have been used to introduce expression vectors into plants. Deshayes et al., EMBO J., 4:2731 (1985), Christou et al., Proc Natl. Acad. Sci. U.S.A. 84:3962 (1987). Direct uptake of DNA into protoplasts using CaCl₂ precipitation, polyvinyl alcohol or poly-L-ornithine has also been reported. Hain et al., Mol. Gen. Genet. 199:161 (1985) and Draper et al., Plant Cell Physiol. 23:451 (1982). Electroporation of protoplasts and whole cells and tissues have also been described. Donn et al., In Abstracts of VIIth International Congress on Plant Cell and Tissue Culture IAPTC, A2-38, p 53 (1990); D'Halluin et al., Plant Cell 4:1495-1505 (1992) and Spencer et al., Plant Mol. Biol. 24:51-61 (1994).

Following transformation of rice target tissues, expression of the above-described selectable marker genes allows for preferential selection of transformed cells, tissues and/or plants, using regeneration and selection methods now well known in the art. The foregoing methods for transformation would typically be used for producing a transgenic cultivar. The transgenic cultivar could then be crossed, with another (non-transformed or transformed) cultivar, in order to produce a new transgenic cultivar. Alternatively, a genetic trait which has been engineered into a particular rice cultivar using the foregoing transformation techniques could be moved into another cultivar using traditional backcrossing techniques that are well known in the plant breeding arts. For example, a backcrossing approach could be used to move an engineered trait from a public, non-elite cultivar into an elite cultivar, or from a cultivar containing a foreign gene in its genome into a cultivar which does not contain that gene. As used herein, “crossing” can refer to a simple X by Y cross, or the process of backcrossing, depending on the context.

### Gene Conversion

When the term rice plant is used in the context of the present invention, this also includes any gene conversions of that cultivar. A gene converted plant refers to those rice plants which are developed by a plant breeding technique called backcrossing wherein essentially all of the desired morphological and physiological characteristics of a cultivar are recovered in addition to the one or more genes transferred into the cultivar via the backcrossing technique. Backcrossing methods can be used with the present invention to improve or introduce one or more characteristics into the cultivar. The term backcrossing as used herein refers to the repeated crossing of a hybrid progeny back to one of the parental rice plants, the recurrent parent, for that cultivar, i.e., backcrossing 1, 2, 3, 4, 5, 6, 7, 8, 9 or more times to the recurrent parent. The parental rice plant which contributes the gene(s) for the desired characteristic(s) is termed the nonrecurrent or donor parent. This terminology refers to the fact that the nonrecurrent parent is used one time in the backcross protocol and therefore does not recur. The parental rice plant to which the gene or genes from the nonrecurrent parent are transferred is known as the recurrent parent as it is used for several rounds in the backcrossing protocol (Poehlman & Sleper, 1994; Fehr, 1987). In a typical backcross protocol, the original cultivar of interest (recurrent parent) is crossed to a second cultivar (nonrecurrent parent) that carries the gene(s) of interest to be transferred. The resulting progeny from this cross are then crossed again to the recurrent parent and the process is repeated until a rice plant is obtained wherein essentially all of the desired morphological and physiological characteristics of the recurrent parent are recovered in the converted plant, in addition to the transferred gene(s) from the nonrecurrent parent.

The selection of a suitable recurrent parent is an important step for a successful backcrossing procedure. The goal of a backcross protocol is to alter or substitute one or more trait(s) or characteristic(s) in the original cultivar. To accomplish this, gene(s) of the recurrent cultivar is/are modified or substituted with the desired gene(s) from the nonrecurrent parent, while retaining essentially all of the rest of the desired genetic, and therefore the desired physiological and morphological, constitution of the original cultivar. The choice of the particular nonrecurrent parent will depend on the purpose of the backcross; one of the major purposes is to add some commercially desirable, agronomically important trait(s) to the plant. The exact backcrossing protocol will depend on the characteristic(s) or trait(s) being altered to determine an appropriate testing protocol. Although backcrossing methods are simplified when the characteristic being transferred is a dominant allele, a recessive allele may also be transferred. In this instance it may be necessary to introduce a test of the progeny to determine if the desired characteristic(s) has been successfully transferred.

Many traits have been identified that are not regularly selected for in the development of a new cultivar but that can be improved by backcrossing techniques. These traits may or may not be transgenic, examples of these traits include but are not limited to, male sterility, waxy starch, herbicide resistance, resistance for bacterial, fungal, or viral disease, insect resistance, male fertility, enhanced nutritional quality, industrial usage, yield stability and yield enhancement. These genes are generally inherited through the nucleus. Some known exceptions to this are the genes for male sterility, some of which are inherited cytoplasmically, but still act as single gene traits. Several of these traits are described in U.S. Pat. Nos. 5,777,196; 5,948,957 and 5,969,212, the disclosures of which are specifically hereby incorporated by reference for this purpose.

### Tissue Culture

Further reproduction of rice plants containing the Rc-g allele can occur by tissue culture and regeneration. Tissue culture of various tissues of rice and regeneration of plants therefrom is well known and widely published. For example, reference may be had to Komatsuda, T. et al., Crop Sci. 31:333-337 (1991); Stephens, P. A., et al., Theor. Appl. Genet. (1991) 82:633-635; Komatsuda, T. et al., Plant Cell, Tissue and Organ Culture, 28:103-113 (1992); Dhir, S. et al., Plant Cell Reports (1992) 11:285-289; Pandey, P. et al., Japan J. Breed. 42:1-5 (1992); and Shetty, K., et al., Plant Science 81:245-251 (1992); as well as U.S. Pat. No. 5,024,944 issued Jun. 18, 1991 to Collins et al., and U.S. Pat. No. 5,008,200 issued Apr. 16, 1991 to Ranch et al. Thus, another aspect of this invention is to provide cells which upon growth and differentiation produce rice plants having the physiological and morphological characteristics of rice plants containing the Rc-g allele.

As used herein, the term “tissue culture” indicates a composition comprising isolated cells of the same or a different type or a collection of such cells organized into parts of a plant. Exemplary types of tissue cultures are protoplasts, calli, plant clumps, and plant cells that can generate tissue culture that are intact in plants or parts of plants, such as embryos, pollen, flowers, seeds, pods, leaves, stems, roots, root tips, anthers, and the like. Means for preparing and maintaining plant tissue culture are well known in the art. By way of example, a tissue culture comprising organs has been used to produce regenerated plants. U.S. Pat. Nos. 5,959,185; 5,973,234 and 5,977,445 describe certain techniques, the disclosures of which are incorporated herein by reference.

As used herein, the term “plant” includes plant cells, plant protoplasts, plant cells of tissue culture from which rice plants can be regenerated, plant calli, plant clumps, and plant cells that are intact in plants or parts of plants, such as pollen, flowers, embryos, ovules, seeds, pods, leaves, stems, pistils, anthers and the like.

The present invention contemplates a rice plant regenerated from a tissue culture of a variety or hybrid plant containing the allele Rc-g of the present invention. As is well known in the art, tissue culture of rice can be used for the in vitro regeneration of a rice plant. Tissue culture of various tissues of rice and regeneration of plants therefrom is well known and widely published. For example, reference may be had to Chu, Q. R., et al., (1999) “Use of bridging parents with high anther culturability to improve plant regeneration and breeding value in rice”, Rice Biotechnology Quarterly 38:25-26; Chu, Q. R., et al., (1998), “A novel plant regeneration medium for rice anther culture of Southern U.S. crosses”, Rice Biotechnology Quarterly 35:15-16; Chu, Q. R., et al., (1997), “A novel basal medium for embryogenic callus induction of Southern US crosses”, Rice Biotechnology Quarterly 32:19-20; and Oono, K., “Broadening the Genetic Variability By Tissue Culture Methods”, Jap. J. Breed. 33 (Suppl.2), 306-307, illus. 1983. Thus, another aspect of this invention is to provide cells which upon growth and differentiation produce rice plants having the physiological and morphological characteristics of rice plants containing allele Rc-g.

Duncan, et al., Planta 165:322-332 (1985) reflects that 97% of the plants cultured that produced callus were capable of plant regeneration. Subsequent experiments with both cultivars and hybrids produced 91% regenerable callus that produced plants. In a further study in 1988, Songstad, et al., Plant Cell Reports 7:262-265 (1988), reports several media additions that enhance regenerability of callus of two cultivars. Other published reports also indicated that “non-traditional” tissues are capable of producing somatic embryogenesis and plant regeneration. K. P. Rao et al., Maize Genetics Cooperation Newsletter, 60:64-65 (1986), refers to somatic embryogenesis from glume callus cultures and B. V. Conger, et al., Plant Cell Reports, 6:345-347 (1987) indicates somatic embryogenesis from the tissue cultures of corn leaf segments. Thus, it is clear from the literature that the state of the art is such that these methods of obtaining plants are routinely used and have a very high rate of success.

Rice varieties containing mutant allele Rc-g of the present invention can also be used for transformation where exogenous genes are introduced and expressed by the variety containing allele Rc-g. Genetic variants created either through traditional breeding methods using a line containing allele Rc-g or through transformation of a line containing allele Rc-g by any of a number of protocols known to those of skill in the art are intended to be within the scope of this invention.

The following describes breeding methods that may be used with a rice plant containing allele Rc-g in the development of further rice plants. One such embodiment is a method for developing a progeny rice plant in a rice plant breeding program comprising: obtaining a rice plant, or a part thereof, which comprises allele Rc-g, utilizing said plant or plant part as a source of breeding material and selecting a progeny plant containing allele Rc-g with molecular markers in common with rice plants containing allele Rc-g and/or with morphological and/or physiological characteristics of rice cultivar Wells. Breeding steps that may be used in the rice plant breeding program include pedigree breeding, back crossing, mutation breeding, and recurrent selection. In conjunction with these steps, techniques such as RFLP-enhanced selection, genetic marker enhanced selection (for example SSR markers) and the making of double haploids may be utilized.

One of ordinary skill in the art of plant breeding would know how to evaluate the traits of two plant varieties to determine if there is no significant difference between the two traits expressed by those varieties. For example, see Fehr and Walt, Principles of Cultivar Development, p 261-286 (1987). Thus the invention includes rice plants containing allele Rc-g progeny rice plants so that said progeny rice plants are not significantly different for said traits than rice plants containing allele Rc-g. Using techniques described herein, molecular markers may be used to identify said progeny plant as a plant containing allele Rc-g progeny plant. Mean trait values may be used to determine whether trait differences are significant, and preferably the traits are measured on plants grown under the same environmental conditions. Once such a variety is developed its value is substantial since it is important to advance the germplasm base as a whole in order to maintain or improve traits such as yield, disease resistance, pest resistance, and plant performance in extreme environmental conditions.

Progeny of rice plants containing allele Rc-g may also be characterized through their filial relationship with rice plants containing allele Rc-g, as for example, being within a certain number of breeding crosses of rice plants containing allele Rc-g. A breeding cross is a cross made to introduce new genetics into the progeny, and is distinguished from a cross, such as a self or a sib cross, made to select among existing genetic alleles. The lower the number of breeding crosses in the pedigree, the closer the relationship between rice plants containing allele Rc-g and its progeny. For example, progeny produced by the methods described herein may be within 1, 2, 3, 4 or 5 breeding crosses of rice plants containing allele Rc-g.

The seed of rice plants containing allele Rc-g, the plant produced from the seed, the hybrid rice plant produced from the crossing of the cultivar, hybrid seed, and various parts of the hybrid rice plant and transgenic versions of the foregoing, can be utilized for human food, livestock feed, and as a raw material in industry

### REFERENCES

1. Arkansas State Plant Board (2002) Official standards for seed certification in Arkansas. Circular 15.
2. Brooks S. A., Yan W, Jackson A. K., Deren C. W. (2008) A natural mutation in rc reverts white-rice-pericarp to red and results in a new, dominant, wild-type allele: Rc-g. Theor Appl Genet. 117:575-580.
3. Furukawa T, Maekawa M, Oki T, Suda I, Iida S, Shimada H, Takamure I, Kadowaki K-i (2007) The Rc and Rd genes are involved in proanthocyanidin synthesis in rice pericarp. Plant J. 49:91-102.
4. Gealy D R, Tai T H, Sneller C H (2002) Identification of red rice, rice, and hybrid populations using microsatellite markers. Weed Sci. 50:333-339.
5. Gramene (2007) Cold Spring Harbor Laboratory and Cornell University, USA. http://www.gramene.org. Cited 26 Dec. 2007.
6. Ji H-S, Chu S-H, Jiang W, Cho Y-I, Hahn J-H, Eun M-Y, McCouch S R, Koh H-J (2006) Characterization and mapping of a shattering mutant in rice that corresponds to a block of domestication genes. Genetics 173: 995-1005.
7. Ling W H, Cheng Q X, Ma J, Wang T (2001) Red and black rice decrease atherosclerotic plaque formation and increase antioxidant status in rabbits. J. Nutr. 131: 1421-1426.
8. Moldenhauer K A K, Lee F N, Bernhardt J L, Norman R J, Slaton N A, Wilson C E, Anders M M, Cartwright R D, Blocker M M (2007) Registration of ‘Wells’ rice. Crop Sci. 47: 442-443.
9. Slaton N, Moldenhauer K, Gibbons J, Blocker M, Wilson C, Dilday R, Robinson J, Koen B (2000) Grain characteristics of rice varieties. University of Arkansas, Cooperative Extension Service, Rice Information Bulletin, No. 146.
10. Sweeney M T, Thomson M J, Pfeil B E, McCouch S (2006) Caught red-handed: Rc encodes a basic helix-loop-helix protein conditioning red pericarp in rice. Plant Cell 18: 283-294.
11. Wilson C (2002) Arkansas rice acreage by variety survey. Proceedings of the 30^{th} Rice Technical Working Group, New Orleans, La.

### DEPOSIT INFORMATION

A deposit of the rice seed containing the Rc-g mutant allele of this invention is maintained by the University of Arkansas, Rice Research and Extension Center, 2900 Hwy. 130 E., Stuttgart, A R 72160. Access to this deposit will be available during the pendency of this application to persons determined by the Commissioner of Patents and Trademarks to be entitled thereto under 37 CFR 1.14 and 35 USC 122. Upon allowance of any claims in this application, all restrictions on the availability to the public of the variety will be irrevocably removed by affording access to a deposit of at least 2,500 seeds of the same variety with the American Type Culture Collection, Manassas, Va.

While a number of exemplary aspects and embodiments have been discussed above, those of skill in the art will recognize certain modifications, permutations, additions and sub-combinations thereof. It is therefore intended that the following appended claims and claims hereafter introduced are interpreted to include all such modifications, permutations, additions and sub-combinations as are within their true spirit and scope.

What is claimed is:

1. A rice seed containing an allele designated Rc-g, wherein a representative sample of seed containing said Rc-g allele has been deposited under ATCC Accession No. PTA-______.

2. A rice plant, or a part thereof, produced by growing the seed of claim 1.

3. A tissue culture of cells or protoplasts produced from the plant of claim 2, wherein said cells or protoplasts of the tissue culture are produced from a plant part selected from the group consisting of leaf, pollen, embryo, cotyledon, hypocotyl, meristematic cell, root, root tip, pistil, anther, flower, stem, glume and panicle.

4. A rice plant regenerated from the tissue culture of claim 3.

5. A method for producing an F₁ hybrid rice seed, wherein the method comprises crossing the plant of claim 2 with a different rice plant and harvesting the resultant F₁ hybrid rice seed.

6. A hybrid rice seed produced by the method of claim 5.

7. A hybrid rice plant, or a part thereof, produced by growing said hybrid seed of claim 6.

8. A method of producing a rice plant with a red pigmented pericarp, wherein the method comprises transforming a rice plant with a transgene encoding a polypeptide having SEQ ID NO: 6.

9. A rice plant having a red-pericarp produced by the method of claim 8.

10. A method of introducing a desired trait into a rice plant containing allele Rc-g, wherein the method comprises:
(a) crossing a rice plant containing allele Rc-g, wherein a representative sample of seed was deposited under ATCC Accession No. PTA-______, with a plant of another rice cultivar that comprises a desired trait to produce progeny plants;
(b) selecting one or more progeny plants that have the desired trait to produce selected progeny plants;
(c) crossing the selected progeny plants with the rice plant containing allele Rc-g to produce backcross progeny plants;
(d) selecting for backcross progeny plants that have the desired trait and the physiological and morphological characteristics of the rice plant containing allele Rc-g to produce selected backcross progeny plants; and
(e) repeating steps (c) and (d) two times to produce selected third or higher backcross progeny plants that comprise the desired trait.

11. A plant produced by the method of claim 10, wherein the plant has the desired trait.

12. The plant of claim 11, wherein the desired trait is a red pigmented pericarp.

13. A rice plant having the Rc-g allele, wherein at least 5% of grains resulting therefrom are red-pigmented.

14. A rice plant having a genotype comprising an allele selected from the group consisting of Rc-g/Rc-g and Rc-g/rc-g.

15. An isolated nucleic acid molecule having SEQ ID NO: 5.

16. The nucleic acid molecule of claim 15, wherein said nucleic acid encodes a polypeptide having SEQ ID NO: 6.

17. The polypeptide of claim 16, wherein said polypeptide has amino acid deletions at positions 466, 467, 468, 469 and 470 and has amino acid substitutions at positions 463, 471, 472 and 474 of SEQ ID NO: 4.

18. An isolated nucleic acid molecule having a one base pair guanine deletion at position 1388 of SEQ ID NO: 3.

19. A method for testing red-pigmented grain for the Rc-g allele comprising:
(a) obtaining a polymerase chain reaction forward primer having SEQ ID NO: 10;
(b) obtaining a polymerase chain reaction reverse primer having SEQ ID NO: 12;
(c) obtaining a red grain sample; and
(d) using said polymerase chain reaction forward and reverse primers to selectively amplify the Rc-g allele in said red grain sample.

20. The method of claim 19, wherein the grain sample is selected from the group consisting of a seed grain sample, a field grain sample, a harvested grain sample, and a bagged grain sample.


SEQUENCE LISTING (13 sequences)

SEQ ID NO: 1 - DNA, 80 bp, Oryza rufipogon
ggggagcagaaacacctgaatcaaggggcgggaaaggcgcaagtggaacgcgaaaagtcggtgccatccaaggtgatttc

SEQ ID NO: 2 - protein, 18 aa, Oryza rufipogon
ESRGGKGASGTRKVGAIQ

SEQ ID NO: 3 - DNA, 66 bp, Oryza sativa
ggggagcagaaacacctgaatcaaggggcgggaaaggcgcaagtggatgccatccaaggtgatttc

SEQ ID NO: 4 - protein, 14 aa, Oryza sativa
ESRGGKGASGCHPR

SEQ ID NO: 5 - DNA, 65 bp, Oryza sativa
ggggagcagaaacacctgaatcaagggcgggaaaggcgcaagtggatgccatccaaggtgatttc

SEQ ID NO: 6 - protein, 13 aa, Oryza sativa
ESRAGKAQVDAIQ

SEQ ID NO: 7 - DNA, 21 bp, Artificial Sequence
  pSNP-white
agaaacacctgaatcaagtgg

SEQ ID NO: 8 - DNA, 58 bp, Oryza rufipogon
agaaacacctgaatcaaggggcgggaaaggcgcaagtggaacgcgaaaagtcggtgcc

SEQ ID NO: 9 - DNA, 44 bp, Oryza sativa
agaaacacctgaatcaaggggcgggaaaggcgcaagtggatgcc

SEQ ID NO: 10 - DNA, 21 bp, Artificial Sequence
  pSNP-red
agaaacacctgaatcaatggc

SEQ ID NO: 11 - DNA, 43 bp, Oryza sativa
agaaacacctgaatcaagggcgggaaaggcgcaagtggatgcc

SEQ ID NO: 12 - DNA, 24 bp, Artificial Sequence
  pSNP-redReverse
gagctcttgtatgcggttccttag

SEQ ID NO: 13 - DNA, 24 bp, Artificial Sequence
  pWhiteReverse
ggatacgggtaggattcacttctg